(19) United States
(12) Patent Application Publication
(10) Pub. No.: US 2002/0127279 A1
(43) Pub. Date: Sep. 12, 2002

(54) Peptide-isolation process and product made thereby

(76) Inventor: James S. Matthews, Circleville, OH (US)

Correspondence Address: SAND & SEBOLT, 4801 DRESSLER RD., N.W., SUITE 194, CANTON, OH, 44718, US

(21) Appl. No.: 10/095,631

(22) Filed: Mar. 11, 2002

Related U.S. Application Data
(60) Provisional application No. 60/274,960, filed on Mar. 12, 2001.

(51) Int. Cl. A61K 35/20

(52) U.S. Cl. 424/535

(57) ABSTRACT

A method of processing colostral whey includes contacting the whey with one or more filters, at least one of which has an average pore size of no more than about 50 nm. During this contacting step, the whey encounters a pressure of no more than about 200 kPa. This contacting step can be preceded by contact with another filter which has an average pore size of no more than about 0.3 m, with this step also being performed at a pressure of no more than about 200 kPa. The result is a filtrate of colostral whey that includes peptides, no more than 10% of which are denatured.

### BACKGROUND OF THE INVENTION

1. Technical Field

The present invention relates to compositions for medical and veterinary use, and more particularly to such compositions that are derived from the colostrum of animals and still more particularly of bovine animals. The present invention also relates to methods for preparing and administering such compositions.

2. Background Information

The present invention relates generally to methods for processing mammalian bodily fluids. Specifically, the present invention relates to methods for separating peptides from such fluids as well as the products of such methods.

For the past quarter century or so, the importance of cellular receptors in various biochemical processes has been recognized and has begun to become better understood. These receptors, which are believed to reside in or on the exterior surfaces of all animal cells (regardless of particular cellular function), are in fact very specialized types of proteins. When activated through interaction with a ligand, a receptor then transmits a biochemical message into the interior of the cell.

Various types of chemical compounds can act as ligands for receptors. One type are small molecules not found naturally in the bodies of animals, e.g., nitrous oxide. Other types of compounds that can act as ligands are synthetic drugs. Other types of material that can enter a receptor are viral components, which often produce undesirable biochemical messages when they bind with one or more receptors.

Those compounds that can act as ligands but which normally are found naturally in animal bodies fall into three general classes: neurotransmitters, steroids (including the sex hormones), and peptides. The first two classes are considered to be bioactive, whereas peptides are not.

Peptides are produced in the ribosomes of all or very nearly all of the cells of animals and man. They generally are relatively small, at least in relation to most proteins; in general, they have a molecular weight of no more than about 1000 daltons although some might be larger, even perhaps as large as about 2500 daltons. Nevertheless, they are on the order of an order of magnitude smaller than most proteins, which tend to have molecular weights of at least 20,000 daltons.

When a peptide leaves the cell in which it was produced, it moves throughout the body by way of the interstitial fluids between the cells and the circulatory system. In the blood and interstitial fluids, peptides tend not to agglomerate with themselves (i.e., they remain separate); this separateness allows the peptides to remain in forms in which they can bind with appropriate receptors.

A peptide produced by one cell can be transported to and interact with the receptor of a distant cell. When such an interaction occurs, a type of biochemical transmission to the cell interior is set into motion and this, in turn, induces some type of a response within the cell. One such cellular action is believed to be the production of additional peptides of the type bound to the cellular receptor.

As mentioned previously, some viruses take up residence in animal bodies by entering cells through particular types of receptors. If the necessary type of receptor already is bound to another ligand, such as a peptide, the virus cannot enter that given cell and must find another cell in which to enter. If all cells have the target receptor bound with other ligands, the virus' entry path is blocked and infection is averted.

When an animal, including a human, is healthy, it has a full (or very nearly full) complement of peptides. However, due to any one or more of a variety of factors, an animal may fail to produce one or more of these peptides. Such failures often can be the first cause of illness. However, return to health can be relatively quick and easy when the missing peptide(s) is reintroduced into the body because such peptides can, as described above, “instruct” cells to create more copies of themselves. Thus, reintroduction of a small amount, perhaps a single copy, of one or more missing peptides can quickly return the body to its normal amount of the peptide(s) in question.

The target peptides can be derived from blood or from mammalian bodily fluids derived from or in contact with blood including, but not limited to, milk, colostrum, semen, urine, vaginal fluid, and the like. However, in materials such as milk and colostrum for example, peptides are in what is essentially an impaired state because they are agglomerated with or on much larger biochemical macromolecules i.e. fats or other proteins. Additionally, ingestion by eating or drinking certainly denatures the peptides because of the acidic conditions of the stomach and the relatively aggressive enzymatic action of the digestive tract.

Thus, although many external sources of peptide ligands are available, these peptides often are in a form that renders them useless for the desired effect. Accordingly, processing or refinement of such external sources is necessary.

Of the external sources of peptides, the one that seems to provide them in the highest concentrations and is most widely available is colostrum. This material has been the subject of numerous processing methodologies. However, almost all of the previously described processing methods appear to have been directed at collecting or isolating biologically active macromolecules that are much larger than peptides such as, for example, proteins, lactoferrin, immunoglobulin, lipids, etc. See, e.g., U.S. Pat. Nos. 4,051,235, 4,377,569, 4,816,252, and the like for further information on such methods.

One exception to this focus on the larger macromolecules as the targeted materials for isolation can be found in U.S. Pat. No. 4,402,938 where the targeted material, termed an “unknown food factor,” has a molecular weight of 1200 daltons and is in the portion of the processed fluid that the other separation processing methods discard. To achieve “the desired economic level” of the material in question, pre-partum introduction of an antigen-like material into the udder of an ungulate is said to be necessary. As peptides do not require the presence of an antigen-like material for their synthesis in the body of humans or other mammals, the likelihood that peptides are the “unknown food factor” described in this reference is low.

Importantly, the colostrum processing methods discussed above either are silent with respect to processing pressures or encourage relatively high pressures as a side effect of fast processing speeds. For those references dealing with ways to isolate large molecules such as immunoglobulin, lactoferrin, etc., this is not surprising because such macromolecules are relatively hearty and capable of withstanding such pressures and, accordingly, are quite different from peptides which can be denatured at pressures ranging from about 210 kPa (˜30 psi) to about 690 kPa (˜100 psi). Those peptides involved in the prevention of viral infections are among those that can be denatured at the lower end of this range of pressures.

A method for processing animal-derived fluids that results in an end product that is peptide-rich but substantially free from other materials that can denature such peptides remains highly desirable.

### BRIEF SUMMARY OF THE INVENTION

The present invention provides means for processing animal-derived fluids so as to provide an end product that is rich in informational peptides (ligands) but substantially free of chemicals and bio-emtities i.e. bacterial that can denature such peptides. Because such peptides must maintain their individual identities and structural integrities to retain their desired effects once introduced into the body, such processing is designed to minimize those agents and effects that tend to denature such peptides.

In a first aspect, a method of processing colostral whey is provided in which the whey is contacted with one or more filters, at least one of which has an average pore size of no more than about 50 nm. During this contacting step, the whey encounters a pressure of no more than about 200 kPa (˜29 psi).

In another aspect, a process of isolating peptides in a fluid of one or more mammals is provided. This process includes removing all macroscopic materials from said fluid to provide a clarified fluid and then contacting the clarified fluid with a first filter having an average pore size of no more than about 0.3 m then with a second filter having an average pore size of no more than about 50 nm. During this process, the peptides encounter a pressure of no more than about 200 kPa (˜29 psi).

In yet another aspect, a process for making a solution or suspension that includes a potable liquid and non-denatured peptides is provided. The process includes mixing the colostrums of more than one mammal so as to provide a colostrum blend; separating and removing the fat from the colostrum blend; curdling the colostrum blend and removing the product of the curdling step so as to provide a whey; contacting the whey with a first filter having an average pore size of no more than about 0.3 m so as to provide a first filtrate that includes the peptides as well as larger proteins; contacting the first filtrate with a second filter having an average pore size of no more than about 50 nm so as to provide a second filtrate which is substantially free of the larger proteins; and collecting the second filtrate. Optionally, the pH of the second filtrate can be adjusted to 4.6. This process is conducted so that the peptides encounter a pressure of no more than about 200 kPa (˜29 psi) throughout the course thereof.

Also provided is a filtrate of colostral whey that includes a potable liquid and peptides. The peptides have molecular weights of no more than 15,000 daltons, and no more than 10% of the peptides are denatured. The filtrate has a pH of from about 4.5 to about 5.0, inclusive.

Unless a contrary intention is expressly indicated, the following definitions are intended to apply herein throughout:

“bioactive” is a phrase that describes a set of compounds which have an effect on animal cells that is in direct proportion to their number and which are produced either outside the body of the animal or only in specialized organs or systems thereof;

“peptide” is a relatively low molecular weight, non-biologically active molecule, produced in the ribosomes of animal cells, which is the product of the sequential, covalent bonding of several (e.g., 4 to about 100) amino acids;

“denatured”, with respect to a peptide, connotes an alteration from the natural state due to, for example, physical forces (e.g., adhesion to another molecule(s), exposure to excessive temperature or pressure during processing, etc.), chemical reaction (e.g., scission due to exposure to excessively acidic or basic conditions), enzymatic degradation, and the like; and

“comprising” means including but not limited to a given set of materials or steps.

### DETAILED DESCRIPTION OF THE INVENTION

Although blood and mammalian bodily fluids derived from or in contact with blood are potential sources for the desired peptides (as described above), colostrum is a preferred source because it has a relatively high level of these peptides (much higher than in milk, for instance) and is produced in relatively large quantities. The first colostrum withdrawn from a given mammal usually has a larger amount of the target peptides (per unit volume) than any subsequent colostrum collected from the same mammal.

For purposes of the following description of the isolation process, colostrum is used as a representative starting material.

Although the production of the target peptides is not affected one way or another, the use of dairy cows which have had their udders specially treated with antibiotics or antigen-like materials is not preferred.

Colostrum is collected and stored under conditions appropriate for the storage of milk for human consumption. Ideally, these conditions are consistent with applicable governmental (e.g., USDA) guidelines. The raw material preferably is collected under conditions that prevent gross contamination by bacteria and immediately placed under refrigeration. If the raw material must be held for more than 48 hours prior to shipment, it preferably is frozen as soon as possible after collection. A primary goal is to protect the target peptides from being denatured by enzymes that can result from the growth of certain types of bacteria.

Since processing typically occurs remote from the point of collection, transportation preferably occurs under conditions appropriate to the handling of milk for human consumption. This can take the form of refrigerated trucks with insulated tanks for large shipments or clean pails with lids for smaller amounts of material.

Specific gravity testing can be performed on random samples or on any shipments that, upon visual inspection, appear to have high amounts of normal milk content. Higher-than-desired milk contents normally do not occur when the amount of colostrum collected from each cow is limited to no more than 15 L (4 gallons). Once determined to be suitable for further processing, the colostrum can be frozen for long term storage, placed under refrigeration for short term storage, or allowed to warm to room temperature for immediate processing. If frozen or refrigerated, the colostrum is warmed just before processing is begun.

Processing can begin with the colostrum in a large vessel, typically a tank made of a relatively inert material such as stainless steel, with some type of stirring mechanism (e.g., a paddle or stirrer blades) to gently mix the raw material. Such mixing helps to counter inconsistencies between colostrums from different animals and to provide a more uniform temperature. Because a given animal, e.g., a cow might be deficient in one or more particular peptides, the blending of colostrums from many cows is preferred so as to ensure that a given colostral blend contains all of the target peptides.

As mentioned previously, when presented with a choice between processing speed and maintaining gentle conditions, the latter is favored. For example, to this point in the process, the material has been kept at or below room temperature (e.g., 25° C). Additionally, where stirrer blades are used as the stirring mechanism in the preceding step, those blades preferably rotate slowly and are angled backward. Where pumps are employed in a given step, they preferably are of a type that do not have impellers or other features capable of producing a shearing effect. In the design of a specific process, each aspect preferably is considered and tailored to prevent damage to the desired peptides.

One of the primary ways that gentle processing conditions are experienced by the desired peptides is to keep the pressure of each part of the process at or below about 200 kPa (˜29 psi), preferably at or below about 175 kPa (˜25 psi), more preferably at or below about 150 kPa (˜22 psi), and most preferably at or below about 140 kPa (˜20 psi). Even the most delicate peptide structures seem to be able to withstand operating pressures of about 140 kPa.

To provide these relatively low operating pressures while maintaining industrially acceptable processing speeds, running the process in the form of a batch (as opposed to continuously) can be preferable. Where batch processing is used, typical amounts can range from about 375 to about 3750 L (100 to 1000 gallons), depending on down-line processing speed.

After blending, the colostrum blend moves to the first phase of the reduction process which involves separating and removing much of the fat. Various means, such as separators and centrifuges, are available to accomplish this task.

The defatted blend then can be conveyed to a curdling vessel (e.g., a stainless steel tank) where it is gently stirred and its temperature raised to between about 32° and about 37° C. (900 to 100° F.) in preparation for curdling. Curdling involves the coagulation of the majority of solids remaining in the blend (principally casein). It generally is accomplished by addition of rennin, an enzyme-rich extract from the stomachs of calves, or an acid such as HCI to the warmed colostrum blend. Once the rennin or acid is added, a curd gradually begins to form soon after stirring of the blend is stopped. As the curd forms, it rises to the surface, producing a soft white cake or crust sitting or floating on whey. The whey, which is the desired product from this step, is drained away from the curdling vessel.

The remaining curd can be cut into small pieces by, for example, activating a stirring mechanism in the tank. The broken curd can be conveyed away from the tank through, for example, stainless steel pipes, to a large screen (also preferably made from a relatively inert material such as stainless steel). Any whey trapped in the curd passes through the screen and, optionally, can be added into the whey collected previously. (The curd can be collected for sale or discarded, as desired.)

The collected whey can be passed through a fines reducer or a clarifier to exclude more of the small pieces of curd that happen to be conveyed away from the curdling vessel during removal of the whey. This additional step, although certainly not required, can be beneficial because it increases the service interval for the filter media described below. The whey is conveyed to the next step of the reduction process through, for example, stainless steel pipes.

The general goal of the next step is to remove those suspended materials that range in size from that which can be seen with the unaided human eye down to just below that which can be seen with a typical optical microscope. Although many separation processes are capable of accomplishing this goal, a tangential flow (sometimes called cross-flow), tubular microfilter unit has been found to be particularly suitable, especially in view of the aforementioned desire to keep operating pressures low. Tangential or cross flow filtration is preferred over dead-end filtration because the latter can result in unacceptable pressures unless throughput speeds and volumes are kept quite low. Additionally, tangential flow units do not result in all particles being trapped in the filter membrane, i.e., certain large particles merely pass along the exterior of the membrane and never get retained in a pore; this extends the operation period for a given filter unit. Microfilter membranes can be made from a wide variety of materials and are commercially available from numerous sources.

Microfilters as a class generally are used to remove substances that range in size from about 0.1 to about 10 m. By selecting a filter with an average pore size of from 0.1 to 0.3 m, one can achieve the desired result of removing most materials having a molecular weight of approximately 500,000 daltons or more, which includes almost all bacteria but very few proteins. In fact, proteins the size of antibodies and smaller pass through this size of filter pore with the greater portion of the water present in the whey.

In the foregoing step, to achieve adequate separation while maintaining operating pressures at or below about 200 kPa (˜29 psi), preferably at or below about 175 kPa (˜25 psi), more preferably at or below about 150 kPa (˜22 psi), and most preferably at or below about 140 kPa (˜20 psi), relatively long filtration units are preferred. These can be on the order of 1 to 2 m in length up to ones that are 10 m or more in length. Membranes for such units can have inner diameters of from 0.05 inch to 2.0 inches and outer diameters of from 1.0 inch to 12.0 inches. Acceptable microfilters for such units include, for example, ceramic filters (polymeric; spiral wound).

The once-filtered liquid is conveyed, again preferably by inert means such as stainless steel pipes, to the next step in the reduction process. Here, the general goal is to remove those suspended materials that range in size down to that which can be seen only with relatively powerful microscopes. Fewer separation processes are capable of accomplishing this goal than were available for the preceding step and, of those, ultrafiltration using a tangential flow unit has been found to be preferred. (Nanofiltration can be used but, due to increased cost and (more importantly) higher operating pressures, ultrafiltration is preferred. Also, as discussed above, tangential flow filtration is preferred to dead-end filtrations due to lesser likelihood of excessive pressures.)

A tangential flow ultrafilter unit is similar in design to the previously described microfilter unit except that its filter has smaller pores. The average pore size of this type of filter ranges from about 5 to about 50 nm, which roughly corresponds to molecules that have molecular weights of from about 5,000 to about 100,000 daltons. A preferred average pore size is from about 7.5 to about 15 nm (corresponding roughly to molecules having molecular weights of from about 15,000 to about 30,000 daltons), with 10 nm being a highly preferred average pore size (corresponding roughly to molecules having molecular weights of about 20,000 daltons). Use of such relatively small pores results of removal of such materials as most proteins (including antibodies), endotoxins, that might have been included in the original colostral blend. The membranes typically are made of cellulosic materials, fluoropolymers, or polysulfones.

As with the other filtration step, the operating pressure is maintained at or below about 200 kPa (˜29 psi), preferably at or below about 175 kPa (˜25 psi), more preferably at or below about 150 kPa (˜22 psi), and most preferably at or below about 140 kPa (˜20 psi); thus, relatively long ultrafiltration units also are preferred. These can be on the order of 1 to 2 m in length up to ones that are 10 m or more in length. Membranes for such units can have inner diameters of from 0.5 inch to 2.0 inches and outer diameters of from 1.0 inch to 12.0 inches. Acceptable ultrafilters for such units include, for example, spiral wound filters (kock; PTI).

In the two preceding reduction (i.e., filtration) steps, the temperature of the filtration units can be maintained as high as about 63° C. (˜145° F.), preferably no more than about 49° C. (˜120° F.), most preferably no more than about 32° C. (˜90° F.). In fact, rather than performing these filtration steps at an elevated temperature, beneficial results have been found from performing one or both, particularly the first one, at reduced temperatures such as, for example, from 0.50 to 5° C. (˜33° to 41° F.), preferably from 1° to 3° C. (˜34° to 37° F.). Use of such relatively low temperatures has been found, for example to keep any fat remaining in the whey in macroscopic globules that do not even enter, and thus occlude, the pores of the filters.

The retentate from the second filtration step contains those antibodies that are the desired products of the prior art processes described previously. Ironically, a byproduct of the present process thus has value and can be frozen and stored for further processing or immediate sale.

Where the ultrafiltration step employs a filter having an average pore size of no more than about 10 nm, the filtrate product is essentially free of biochemical macromolecules that have a molecular weight of 20,000 daltons or greater. This is important for several reasons, one of which is that the filtrate then is substantially free of molecules that can act as points of agglomeration for the desired peptides. A peptide that has agglomerated to other peptides or to larger proteins usually is denatured. Likewise, peptides subject to bacterial-induced enzymatic degradation also can be denatured and, accordingly, the filtrate product preferably is essentially free of such bacteria.

The filtrate product preferably has no more than about 10%, more preferably no more than about 5%, of its component peptides in a denatured state.

The filtrate product preferably is essentially free of peptides or proteins having molecular weights of about 20,000 daltons or greater, more preferably is essentially free of peptides or proteins having molecular weights of about 10,000 daltons or greater, and most preferably has at least 90% of its component peptides having molecular weights of no more than about 2500 daltons.

The filtrate product can be slightly acidified by adding a relatively mild acid such as, for example, citric acid such that the resulting pH of the product is in the range of from about 4.5 to about 5.0, preferably of from about 4.55 to about 4.70, and most preferably of about 4.6. This slightly acidic pH has been found to be sufficient to kill or disable any bacterium that might have made its way through the processing or that might have been in the container used to hold the filtrate product.

Each milliliter of filtrate product preferably includes at least 0.3 g of total protein, more preferably at least 0.35 g protein. Most preferably, each milliliter of product contains from 0.36 to 0.38 g of the peptides. Although this amount seems small, it has been found to provide a full (or very nearly full) array of target peptides, thus providing maximum benefit to the human or animal which receives a dose thereof.

The filtrate product can be administered to maintain wellness or to induce recovery from a wide range of infectious and progressive disease processes. The product preferably is administered as an oral spray. (For adults, the composition is typically administered twice a day wherein each administration consists of five successive 1 mL sprays; for children, the composition is preferably administered twice a day wherein each administration consists of two or three 1 mL sprays; for infants, the composition preferably is administered twice a day wherein each administration consists of one 1 mL spray.) Other administration routes include, without limitation, injection, topical application, intraocular application, nebulization or atomization, and the like. For example, a topical application of the composition might be indicated when treating a burn or wound.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of the invention is an example and the invention is not limited to the exact details shown or described.

1. A method of processing colostral whey, said method comprising contacting said whey with one or more filters, at least one of said one or more filters having an average pore size of no more than 50 nm, said whey encountering a pressure during the contacting step of no more than 200 kPa.

2. The method of claim 1 wherein said colostrum is collected from one or more cows.

3. The method of any of claims 1 wherein said contacting step is performed at a temperature of from 0.5° to 5° C.

4. The method of any of claims 1 wherein said contacting step comprises allowing said whey to contact
a) a first filter having an average pore size of no more than 0.3 m, a nd
b) a second filter having an average pore size of no more than 50 nm.

5. The method of claim 4 wherein said first filter has an average pore size of from 0.1 to 0.3 m.

6. The method of any of claims 4 wherein said second filter has an average pore size of from 5 to 50 nm.

7. The method of claim 6 wherein said second filter has an average pore size of from 7.5 to 15 nm.

8. The method of any of claims 1 wherein said whey encounters a pressure of no more than 175 kPa.

9. The method of any of claims 1 wherein said whey encounters a pressure of no more than 150 kPa.

10. The method of any of claims 1 wherein said whey encounters a pressure of no more than 140 kPa.

11. A process of isolating peptides in a fluid of one or more mammals, comprising:
a) removing all macroscopic materials from said fluid to provide a clarified fluid; and
b) contacting said clarified fluid with
i) a first filter having an average pore size of no more than 0.3 m, a nd
ii) a second filter having an average pore size of no more than 50 nm, said peptides encountering a pressure during the process of no more than 200 kPa.

12. The process of claim 11 wherein said fluid is colostrum.

13. The process of any of claims 11 wherein said one or more mammals comprises a cow.

14. The process of any of claims 11 wherein the contacting steps are performed at a temperature of from 0.5° to 5° C.

15. The process of any of claims 11 wherein said first filter has an average pore size of from 0.1 to 0.3 m.

16. The process of any of claims 11 wherein said second filter has an average pore size of from 5 to 50 nm.

17. The process of claim 16 wherein said second filter has an average pore size of from 7.5 to 15 nm.

18. The process of any of claims 11 wherein said peptides encounter a pressure of no more than 175 kPa.

19. The process of any of claims 11 wherein said peptides encounter a pressure of no more than 150 kPa.

20. The process of any of claims 11 wherein said peptides encounter a pressure of no more than 140 kPa.

21. A process for making a solution or suspension comprising a potable liquid and non-denatured peptides, said process comprising:
a) mixing the colostrums of more than one mammal so as to provide a colostrum blend;
b) separating and removing the fat from said colostrum blend;
c) curdling said colostrum blend and removing the product of the curdling step so as to provide a whey;
d) contacting said whey with a first filter having an average pore size of no more than 0.3 m so as to provide a first filtrate comprising said peptides and larger proteins;
e) contacting said first filtrate with a second filter having an average pore size of no more than 50 nm so as to provide a second filtrate which is substantially free of said larger proteins;
f) collecting said second filtrate and, optionally, adjusting the pH thereof to 4.6, said process being conducted so that said peptides encounter a pressure of no more than 200 kPa throughout the course thereof.

22. The process of claim 21 wherein said colostrum is derived, at least in part, from a cow.

23. The process of any of claims 21 wherein said process is conducted such that said peptides encounter a temperature of no more than 65° C. throughout the course thereof.

24. The process of any of claims 21 wherein said first filter has an average pore size of from 0.1 to 0.3 m.

25. The process of any of claims 21 wherein said second filter has an average pore size of from 5 to 50 nm.

26. The process of claim 25 wherein said second filter has an average pore size of from 7.5 to 15 nm.

27. The process of any of claims 21 wherein said peptides encounter a pressure of no more than 175 kPa.

28. The process of any of claims 21 wherein said peptides encounter a pressure of no more than 150 kPa.

29. The process of any of claims 21 wherein said peptides encounter a pressure of no more than 140 kPa.

30. The process of any of claims 21 wherein said process is conducted in a batch format.

31. A filtrate of colostral whey, comprising:
a) a potable liquid,
b) peptides having molecular weights of no more than 15,000 daltons, no more than 10% of said peptides being denatured, said filtrate having a pH of from 4.5 to 5.0, inclusive.

32. The filtrate of claim 31 wherein each of said peptides has a molecular weight of no more than 10,000 daltons.

33. The filtrate of claim 31 wherein at least 90% of said peptides have molecular weights of no more than 2500 daltons.

34. The filtrate of any of claims 31 wherein no more than 5% of said peptides are denatured.

35. The filtrate of any of claims 31 wherein said filtrate is essentially free of biochemical macromolecules having a molecular weight of at least 20,000 daltons.

36. The filtrate of any of claims 31 having a pH of from 4.55 to 4.70.

37. The filtrate of any of claims 31 wherein said filtrate is essentially free of bacteria capable of denaturing total protein.

38. The filtrate of any of claims 31 wherein each milliliter of liquid comprises at least 0.3 g of total protein.

39. The filtrate of any of claims 31 wherein each milliliter of liquid comprises at least 0.35 g of total protein.

40. The filtrate of any of claims 31 wherein each milliliter of liquid comprises from 0.36 to 0.38 g of said peptides.
